(19) United States
(12) Patent Application Publication
(10) Pub. No.: US 2010/0159021 A1
(43) Pub. Date: Jun. 24, 2010

(54) Small Molecule Ligands of the Integrin RGD Recognition Site and Methods of Use

(76) Inventors: Paul Davis, West Sand Lake, NY (US); Faith B. Davis, West Sand Lake, NY (US); Shaker A. Mousa, Wynantskill, NY (US); Hung-Yun Lin, Schenectady, NY (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/644,493

(22) Filed: Dec. 22, 2009

Related U.S. Application Data
(60) Provisional application No. 61/140,223, filed on Dec. 23, 2008.

(51) Int. Cl. A61K 47/48; A61K 31/192; A61K 31/05; A61K 9/14; A61K 31/138; A61P 35/00

(52) U.S. Cl. 424/499; 514/570; 514/733; 424/489; 514/648; 424/501

(57) ABSTRACT

Provided herein are compositions and methods for treating cancer by increasing the pro-apototic actions of small molecule ligands of integrin RGD recognition sites such as polyphenols by administering such compounds in conjunction with anti-angiogenic thyroid hormone analogs such as tetrac or triac.

### RELATED APPLICATIONS

This application claims priority to U.S. Ser. No. 61/140,223, filed Dec. 23, 2008, which is herein incorporated by reference in its entirety.

### FIELD OF THE INVENTION

The invention described herein pertains tetraiodothyroacetic acid-like compounds and other small molecules that bind to the Arg-Gly-Asp (RGD) recognition site on plasma membrane integrins as well as various formulations thereof and methods for the prevention or treatment of cancer.

### BACKGROUND

Thyroid hormones, such as L-thyroxine (T4) and 3,5,3′-triiodo-L-thyronine (T3), and their analogs such as GC-1, DITPA, Tetrac and Triac, regulate many different physiological processes in different tissues in vertebrates. It was previously known that many of the actions of thyroid hormones are mediated by the thyroid hormone receptor (“TR”). However, a novel cell surface receptor for thyroid hormone (L-thyroxine, T4; T3) has been described on integrin αvβ3. This receptor is at or near the Arg-Gly-Asp (RGD) recognition site on the integrin. The αvβ3 receptor is not a homologue of the nuclear thyroid hormone receptor (TR), but activation of this cell surface receptor results in a number of nucleus-mediated events, including the recently-reported pro-angiogenic action of the hormone and fibroblast migration in vitro in the human dermal fibroblast monolayer model of wound-healing.

Tetraiodothyroacetic acid (tetrac) is a deaminated analog of T4 that has no agonist activity at the integrin, but inhibits binding of T4 and T3 by the integrin as well as the pro-angiogenic action of agonist thyroid hormone analogs at αvβ3. Inhibition of the angiogenic action of thyroid hormone by tetrac has been shown in the chick chorioallantoic membrane (CAM) model and in the vessel sprouting model involving human dermal microvascular endothelial cells (HDMEC). In the absence of thyroid hormone, tetrac blocks the angiogenic activity of basic fibroblast growth factor (bFGF, FGF2), vascular endothelial growth factor (VEGF), and other pro-angiogenic peptides. Tetrac is effective in the CAM and HDMEC models. This inhibitory action of tetrac is thought to reflect its influence at the RGD recognition site on the integrin, which is relevant to cell surface pro-angiogenic growth factor receptors with which the integrin engages in cross talk and whose activities may be modulated by the integrin.

Evidence that thyroid hormone can act primarily outside the cell nucleus has come from studies of mitochondrial responses to T3 and diiodothyronine (T2), from rapid onset effects of the hormone at the cell membrane and from actions on cytoplasmic proteins. The recent description of a plasma membrane receptor for thyroid hormone on integrin αvβ3 has provided some insight into effects of the hormone on membrane ion pumps, such as the Na+/H+ antiporter, and has led to the description of interfaces between actions initiated at the membrane thyroid hormone receptor and nuclear events that underlie important cellular or tissue processes, such as, for example, angiogenesis and proliferation of certain tumor cells.

Circulating levels of thyroid hormone are relatively stable. Therefore, membrane-initiated actions of thyroid hormone on neovascularization, on cell proliferation, or on membrane ion channels—as well, of course, as gene expression effects of the hormone mediated by TR mentioned above—may be assumed to contribute to ‘basal activity’ or setpoints of these processes in intact organisms. The possible clinical utility of those cellular events that are mediated by the membrane receptor for thyroid hormone may reside in the inhibition of such effect(s) in the contexts of neovascularization or tumor cell growth. Indeed, blocking the membrane receptor for iodothyronines with tetraiodothyroacetic acid (tetrac), a hormone-binding inhibitory analog that has no agonist activity at the receptor, can arrest growth of glioma cells and of human breast cancer cells in vitro. Thus, tetrac is a useful probe to screen for participation of the integrin receptor in actions of thyroid hormone.

Integrin αvβ3 binds thyroid hormone near the Arg-Gly-Asp (RGD) recognition site of the integrin protein. The RGD site is involved in the protein-protein interactions linking the integrin to extracellular matrix (ECM) proteins such as vitronectin, fibronectin and laminin. (See Plow et al. J. Biol. Chem. 275:21785-88, 2000). Also initiated at the cell surface integrin receptor is the complex process of angiogenesis, which can be monitored in either a standard chick blood vessel assay or with human endothelial cells in a sprouting assay. This hormone-dependent process requires mitogen-activated protein kinase (MAPK; extracellular regulated kinase [ERK] 1/2) activation and the elaboration of vascular growth factors, including, but not limited to basic fibroblast growth factor (bFGF; FGF2), which is the downstream mediator of thyroid hormone's effect on angiogenesis. Tetrac blocks this action of T4 and T3, as does RGD peptide and other small molecules (such as XT-199) that mimic RGD peptide(s). Thus, it is possible that desirable neovascularization can be promoted with local application of thyroid hormone analogs, for example, in wound-healing, or that undesirable angiogenesis, such as that which supports tumor growth, can be antagonized with tetrac.

### SUMMARY OF THE INVENTION

The invention provided pharmaceutical compositions for treating cancer involving a combination of resveratrol and tetrac, where the combination induces apoptosis in cancer cells. Further, the resveratrol and/or the tetrac can be used in a nanoparticulate form. When in nanoparticulate form, the nanoparticulates may also target additional chemotherapeutic agents to the cancer cells. Additionally, in various embodiments, the composition also contains an anti-estrogen compound. Moreover, the combination of resveratrol and tetrac can be used to block the anti-apoptotic action of endogenous thyroid hormone.

More specifically, provided herein are pharmaceutical compositions for treating cancer that contain a combination of small molecule ligand of the integrin RGD recognition site and an anti-angiogenic thyroid hormone analog, wherein the combination induces apoptosis in cancer cells. For example, the cancer cells may be selected from breast cancer, lung cancer, kidney cancer, thyroid cancer, brain cancer (glioma), ovarian cancer, pancreatic cancer, prostate cancer, plasma cell cancer (myeloma), squamous cell head-and-neck cancer, liver cancer, muscle cancer (sarcoma), and colon cancer.

In various embodiments described herein, the small molecule ligand of the integrin RGD recognition site is obtained or derived from a polyphenol compound and the thyroid hormone analog is tetrac or triac (triiodothyroacetic acid). By way of non-limiting example, the small molecule ligand of the integrin RGD recognition site that is used in the compositions of the invention is resveratrol. One preferred the thyroid hormone analog that is used in the compositions of the invention is tetrac.

In any of the pharmaceutical compositions described herein, resveratrol and/or or tetrac are conjugated via a covalent bond to a polymer selected from polyvinyl alcohol, acrylic acid ethylene co-polymer, polyethyleneglycol (PEG), polyacrylic acid, polylactic acid, agarose, polyglycolide, polylactide, PEO, m-PEG, PVA, PLLA, PGA, poly-L-lysine, Human Serum Albumin, cellulose derivatives, carbomethoxy/ethyl/hydroxypropyl, hyaluronic acid, folate linked cyclodextrin/dextran, sarcosine/amino acid spaced polymer, alginate, carrageenan, pectin/chitosan, chitosan, dextran, collagen, polyamine, poly aniline, poly alanine, polytrytophan, poly tyrosine, polylactide-co-glycolide (PLGA) polyglycolide, polylactic acid, or co-polymers thereof, wherein the polymer is formulated into a nanoparticle, wherein the nanoparticle is between 150 and 250 nanometers in size, and wherein the tetrac binds to the cell surface receptor for thyroid hormone on integrin αvβ3.

The resveratrol can also be formulated as any of the conjugates depicted in FIGS. 4-7.

The pharmaceutical compositions may also contain one or more anti-estrogen compounds (e.g., tamoxifen and/or aromatase inhibitors).

Alternatively (or additionally), the nanoparticles may also contain one or more additional chemotherapeutic agents. In such embodiments, the one or more additional chemotherapeutic agents are targeted to the cancer cells.

Those skilled in the art will recognize that the combination of a small molecule ligand of the integrin RGD recognition site and an anti-angiogenic thyroid hormone analog blocks anti-apoptotic action of endogenous thyroid hormone.

Also provided herein are methods of treating cancer comprising administering a therapeutically effective amount of a combination of a small molecule ligand of the integrin RGD recognition site and an anti-angiogenic thyroid hormone analog to a patient suffering therefrom. In various embodiments, the small molecule ligand of the integrin RGD recognition site is obtained or derived from a polyphenol compound and the thyroid hormone analog is tetrac or triac. In one preferred embodiment, the small molecule ligand of the integrin RGD recognition site is resveratrol, and the thyroid hormone analog is tetrac.

In any of the methods disclosed herein, resveratrol and/or tetrac are conjugated via a covalent bond to a polymer selected from polyvinyl alcohol, acrylic acid ethylene co-polymer, polyethyleneglycol (PEG), polyacrylic acid, polylactic acid, agarose, polyglycolide, polylactide, PEO, m-PEG, PVA, PLLA, PGA, poly-L-lysine, Human Serum Albumin, cellulose derivatives, carbomethoxy/ethyl/hydroxypropyl, hyaluronic acid, folate linked cyclodextrin/dextran, sarcosine/amino acid spaced polymer, alginate, carrageenan, pectin/chitosan, chitosan, dextran, collagen, polyamine, poly aniline, poly alanine, polytrytophan, poly tyrosine, polylactide-co-glycolide (PLGA) polyglycolide, polylactic acid, or co-polymers thereof, wherein said polymer is formulated into a nanoparticle, wherein said nanoparticle is between 150 and 250 nanometers in size, and wherein said tetrac binds to the cell surface receptor for thyroid hormone on integrin αvβ3.

Resveratrol can also be formulated as any of the conjugates depicted in FIGS. 4-7.

Additionally, the methods of the invention may also involve administering one or more anti-estrogen compounds to the subject. By way of non-limiting example, the anti-estrogen compounds are tamoxifen and/or aromatase inhibitors.

Moreover, the nanoparticles used in the methods of the invention may also contain one or more additional chemotherapeutic agents.

Those skilled in the art will recognize that the nanoparticles disclosed herein can be used to induce chemosensitization of chemoresistant cancer cells. Thus, these nanoparticles can be used to suppress the development of chemotherapeutic drug resistance in cancer cells.

Likewise, the nanoparticles can also be used to induce radiosensitivity or oppose radioresistence of cancer cells. In general, radiosensitivity is the relative susceptibility of cells, tissues, organs or organisms to the harmful effect of ionizing radiation. Cells are least sensitive when in the S phase, then the G₁ phase, then G₂ phase and the most sensitive in the M phase of the cell cycle. Overall, X-rays are more effective on cells which have a greater reproductive activity.

### BRIEF DESCRIPTION OF THE DRAWINGS

FIG. 1 shows representative polyphenols, of which family resveratrol (FIG. 4) is a member. These are naturally-occurring compounds (i.e., genistein in soy, quercetin in onions, kaempferol in broccoli) some of which are already known to have anti-cancer properties, that are suitable for conjugation to polymers and for cancer chemotherapeutic applications, whether unmodified or formulated as polymers, in conjunction with tetrac and derivatives of tetrac. A polyphenol receptor site in the RGD recognition domain has been described on integrin αvβ3 on cancer cell (see H Y Lin et al., FASEB J20:1742-1744, 2006). The receptor site is close to, but does not interact with, the tetrac-thyroid hormone receptor on the integrin. (See J J Bergh et al., Endocrinology 146:2864-2871, 2005).

FIG. 2 shows representative synthetic, cyclic Arg-Gly-Asp (RGD) analogs that probe the functions of the RGD recognition site on certain integrins (particularly αvβ3) and are antagonists of functions of the integrin that involve the RGD recognition site. These analogs are ligands of the RGD recognition site and interfere with the interactions of natural, extracellular matrix polypeptide ligands of the integrin, including, but not limited to, vitronectin, fibronectin and osteopontin, as well as certain growth factors, all of which contain RGD sequences.

FIG. 3 shows additional synthetic cyclic RGD analogs that serve as ligands of the RGD recognition site on certain integrins (particularly αvβ3) and are antagonists of functions of the integrin that involve the RGD recognition site.

FIG. 4 depicts polymeric conjugates of polyphenolic compounds, including, but not limited to, resveratrol, which are intended for application as anti-cancer ligands of the integrin receptor site for polyphenols in the RGD recognition domain. Polymeric formulation of these compounds limits activity of the polyphenol to the cell exterior and the integrin receptor. Thus, such formulations of polyphenols, including, but not limited to, resveratrol are not subject to cellular uptake and to metabolism.

FIG. 5 shows the synthesis of a polyphenolic polymer, including, but not limited to, catechin converted to an mPEG polymer.

FIG. 6 depicts the structures of additional polymer conjugates, namely, a poly-(lactide-co-glycolide) (PLGA)-immobilized formulation (upper panel) or a hyaluronic acid chain polymer (lower panel).

FIG. 7 shows formulation of a polyphenol polymer (e.g., catechin or epicatechin) as well as a protein-bound polyphenol. Again, these formulations limit activity of the polyphenol to the cell exterior and the integrin receptor, and these formulations of the polyphenols are not subject to cellular uptake and metabolism.

FIGS. 8A and 8B depict the anti-proliferative activities of unmodified tetrac, unmodified resveratrol (RV) and the combination thereof against a human breast cancer (MDA-MB-231) cell line in vitro. The cell line is estrogen receptor (ER)-negative. The test system is a CESCO bellows bottle perfusion model in which cancer cells adhere to plastic flakes and are then exposed to continuous, recycled flow of chemotherapeutic agent in an oxygenated environment. Polyphenol (10 μM) or tetrac (0.1 μM) in DMEM buffer, supplemented with fetal bovine serum, are refreshed daily. The combination of the polyphenol (in this example, resveratrol) and tetrac completes arrests cell proliferation and is more effective than either agent, alone. Results shown are means of triplicate experiments.

FIG. 9 shows the signature on RNA microarray of 50 differentially-regulated genes of MDA-MB-231 cells treated for 24 h with unmodified resveratrol (10 μM). The action of the agent on gene expression is initiated at the cell surface integrin receptor for polyphenols. Pro-apoptotic genes, such as CASP2 and NLRP1 (NALP1), are upregulated by resveratrol, whereas tumor markers, such as FTCD (hepatoma) and the leukemogenic FUS gene, are suppressed.

### DETAILED DESCRIPTION

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural references unless the context clearly dictates otherwise. All patents and publications cited in this specification are incorporated by reference in their entirety.

For convenience, certain terms used in the specification, examples and claims are collected here. Unless otherwise defined, all technical and scientific terms used herein have the same meaning as commonly understood by one of ordinary skill in the art to which this invention pertains.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

As used herein, the terms “angiogenesis agent” or “angiogenic agent” include any compound or substance that promotes or encourages angiogenesis, whether alone or in combination with another substance. Examples include, but are not limited to, T3, T4, T3 or T4-agarose, polymeric analogs of T3, T4, 3,5-dimethyl-4-(4′-hydroy-3′-isopropylbenzyl)-phenoxy acetic acid (GC-1), or DITPA. In contrast, the terms “anti-angiogenesis agent” or “anti-angiogenic agent”, as used herein, refer to any compound or substance that inhibits or discourages angiogenesis, whether alone or in combination with another substance. Examples include, but are not limited to, tetrac, triac, XT 199, and mAb LM609. The structures of representative angiogenic and anti-angiogenic agents are provided herein:

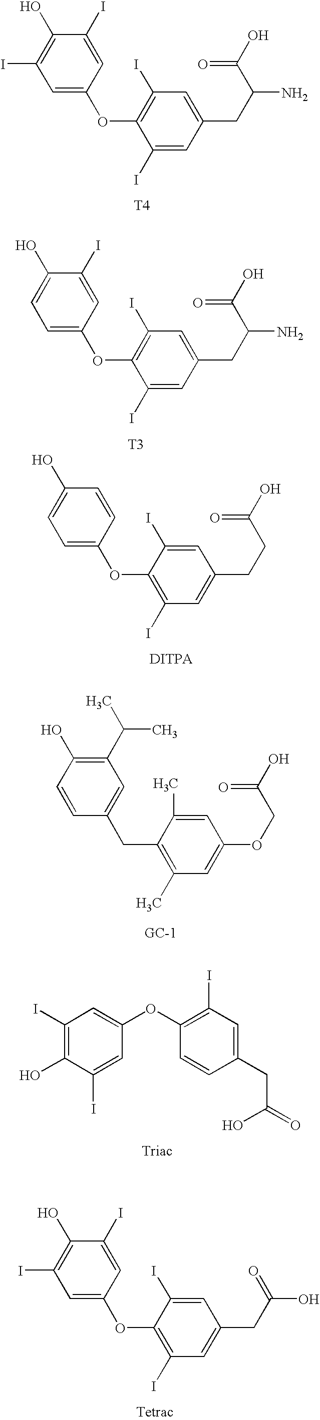

The term “RGD”, as used herein, refers to the single letter amino acid code and references the tripeptide amino acid sequence arginine-glycine-aspartic acid (Arg-Gly-Asp).

The terms “RGD-binding compound” and “small molecule ligand of the integrin RGD recognition site” are used herein to refer to a compound that modulates at least one activity of a protein having the amino acid sequence Arg-Gly-Asp.

A “small molecule” or “small molecule chemical compound” as used herein, is meant to refer to a composition that has a molecular weight of less than 2000 Daltons, preferably less than 1000 Daltons, more preferably less than 750 Daltons, most preferably less than 500 Daltons. Small molecules are organic or inorganic and are distinguished from polynucleotides, polypeptides, carbohydrates and lipids.

The terms “peptide mimetic”, “mimetic”, or “peptidomimetic” as used herein refer to a compound that mimics at least one activity of a peptide or compound or a peptide analog in which one or more peptide bonds have been replaced with an alternative type of covalent bond that is not susceptible to cleavage by peptidases. For example, an RGD-binding compound mimetic refers to a compound that mimics a compound that modulates at least one activity of a protein encoding the amino acid sequence Arg-Gly-Asp.

When referring to a compound, a “form that is naturally occurring” means a compound that is in a form, e.g., a composition, in which it can be found naturally. For example, since resveratrol is found in red wine, this compound is present in red wine in a form that is naturally occurring. A compound is “not in a form that is naturally occurring” if, for example, the compound has been purified and separated from at least some of the other molecules that are typically found with the compound in nature. Thus, a “naturally occurring compound” refers to a compound that can be found in nature, i.e. a compound that has not been designed by man. A naturally occurring compound may be harvested from nature or refined from a complex mixture of naturally occurring products or it may be reproduced synthetically.

As used herein, “sirtuin protein” refers to a member of the sirtuin deacetylase protein family or preferably to the Sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106), and SIRT2 (GenBank Accession No. NM_{—}030593 and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRTS, hSIRT6 and hSIRT7 (see Brachmann et al. Genes Dev. 9:2888, 1995, and Frye et al. BBRC 260:273, 1999). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has. Those skilled in the art will recognize that SIRT1 is a natural target of resveratrol.

A “patient,” “individual,” “subject” or “host” refers to either a human or a non-human animal.

The term “modulation” is art-recognized and refers to up regulation (i.e., activation or stimulation), down regulation (i e , inhibition or suppression) of a response, or any combination thereof.

The terms “prophylactic” or “therapeutic” treatment are art-recognized and refer to the administration of one or more drugs or compounds to a host. If administration occurs prior to clinical manifestation of the unwanted condition (e.g., disease or other unwanted state of the host animal), the treatment is prophylactic, i.e., it protects the host against developing the unwanted condition. If administration occurs after the manifestation of the unwanted condition, the treatment is therapeutic, i.e., it is intended to diminish, ameliorate or maintain the existing unwanted condition or other side effects.

The term “mammal” is known in the art and includes humans, primates, bovines, porcines, canines, felines, and rodents (e.g., mice and/or rats).

As used herein, the term “pharmaceutically-acceptable salt” is art-recognized and refers to the relatively non-toxic, inorganic and organic acid addition salts of compounds, including, for example, those contained in the compositions described herein.

The term “pharmaceutically acceptable carrier” is art-recognized and refers to a pharmaceutically-acceptable material, composition or vehicle, such as, for example, a liquid or solid filler, diluent, excipient, solvent or encapsulating material, involved in carrying or transporting any subject composition or component thereof from one organ or portion of the body, to another organ or portion of the body. Each carrier must be “acceptable” in the sense of being compatible with the subject composition and its components and not injurious to the patient. Some non-limiting examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally”, as used herein, are all art-recognized and refer to the administration of a subject composition, therapeutic or other material other than directly into the central nervous system, such that it enters the patient's system and, thus, is subject to metabolism and other like processes.

Likewise, the terms “parenteral administration” and “administered parenterally” are also art-recognized and refer to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and/or intrasternal injection and infusion.

As used herein, “treating” a condition or disease refers to curing as well as ameliorating at least one symptom of the condition or disease.

The term “therapeutic agent” is art-recognized and refers to any chemical moiety that is a biologically, physiologically, or pharmacologically active substance that acts locally or systemically in a subject. This term also refers to any substance intended for use in the diagnosis, cure, mitigation, treatment or prevention of disease or in the enhancement of desirable physical or mental development and/or conditions in an animal or human.

Moreover, the term “therapeutic effect” is art-recognized and refers to a local or systemic effect in animals, particularly mammals, and more particularly humans, caused by a pharmacologically active substance. The phrase “therapeutically-effective amount” means that amount of such a substance that produces some desired local or systemic effect at a reasonable benefit/risk ratio and is applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art. For example, certain compositions described herein may be administered in a sufficient amount to produce a desired effect at a reasonable benefit/risk ratio applicable to such treatment.

The term “synthetic” is art-recognized and refers to production by in vitro chemical or enzymatic synthesis.

Throughout this application, the terms “nanoparticle”, “nanoparticulate” and “nanoparticulate form” are used interchangeably to refer to a modification of any of the active compound(s) of the invention (i.e., the RGD-binding compounds and/or the anti-angiogenic thyroid hormone analogs (i.e., tetrac and/or triac)), where the active compound(s) are covalently bound (e.g., by an ester, ether, or sulfur linkage) to a polymer wherein the polymer is formulated into a nanoparticle, wherein the active compound is located on the surface of the nanoparticle and wherein the nanoparticle is between 150 and 250 nm in size. Conjugation of the small molecule ligand of the RGD-recognition site on the integrins and/or the anti-angiogenic thyroid hormone analogs via covalent bond to a polymer increases the half life of the compound and/or insures that the compound does not gain access to the interior of the cells (thus, limiting their action to the integrin binding site). The preparation and use of nanoparticulate forms of the anti-angiogenic thyroid hormone analogs are described in the art (see, e.g., WO2008/140507, which is herein incorporated by reference in its entirety). In addition, those skilled in the art will be able to routinely prepare nanoparticulate forms of small molecule ligands of the RGD recognition site on integrins.

Resveratrol is a naturally-occurring polyphenol that has been reported to retard aging in lower animals (see J A Baur, D A Sinclair, Nat Rev Drug Discov 5:493-506, 2006, incorporated herein by reference) and to improve insulin sensitivity (see J A Baur et al. Nature 444:337-342. 2006, incorporated herein by reference).

An extensive literature search from a number of laboratories documents the anti-cancer activity of resveratrol. This polyphenol induces programmed cell death (apoptosis) in a variety of cancer cells by one or more very complex signal transduction mechanisms (see H Y Lin et al. J Cell Biochem, 104:2131-2142, 2008, incorporated herein by reference). A cell surface receptor for resveratrol has also been described by H Y Lin et al. at Ordway (FASEB J 20:1742-1744, 2006, incorporated herein by reference). When activated by resveratrol, this receptor initiates the transduction of the polyphenol signal into apoptosis via the mitogen-activated protein kinase (MAPK; extracellular regulated kinase 1/2 [ERK1/2]) cascade. Others have shown that integrin αvβ3 is expressed on tumor cells, on endothelial and vascular smooth muscle cells, and on osteoclasts (see Davis et al. Front Neuroendocrinol 29:211-18, 2008). This limited expression makes this integrin an attractive target for the development of cancer treatment strategies and also makes it possible to limit the side effect profile of agents directed at the resveratrol receptor site on the integrin. In addition to pro-apoptotic actions on cancer cells, resveratrol has anti-angiogenic activity (see M H Oak et al, J Nutr Biochem 16:1-8, 2005; Mousa et al. Nutr Cancer 52:59-65, 2005)) that is relevant to inhibition of proliferating vasculature that serves tumors.

Thyroid hormone is anti-apoptotic via its action on integrin αvβ3 and, thus, opposes the pro-apoptotic actions of resveratrol. Moreover, tetrac blocks the anti-apoptotic effect of endogenous thyroid hormone. Therefore, combination treatment with resveratrol and tetrac, the actions of both of which are directed at the integrin, is proposed. Such a combination has an additional advantage of summated anti-angiogenic actions.

RGD is the single letter amino acid code for arginine-glycine-aspartate. This tripeptide motif can be found in proteins of the extracellular matrix. For example, integrins (e.g., integrin αvβ3) link the intracellular cytoskeleton of cells to the extracellular matrix that is dependent upon interactions of integrins with RGD sequence-containing extracellular matrix proteins, such as vitronectin and fibronectin. (See Plow et al. J. Biol Chem 275:21785-21788, 2000). Soluble RGD peptides inhibit cell attachment to extracellular proteins, and, thus, may be useful as drugs against angiogenesis (see Kim et al. J Control Release 106:224-234, 2005), inflammation (see Yu et al. J Invest Dermatol 117:1554-1558, 2001), and/or cancer metastasis (see Mandelian et al. Cancer 115:1753-64, 2009).

Provided herein are RGD-binding compounds useful for treating cancer. Exemplary compounds include, but are not limited to, various polyphenols either alone or conjugated to one or more polymers. Polyphenols can include, for example, resveratrol, fisetin, butein, piceatannol, quercetin, and analogs thereof In a preferred embodiment, the polyphenol is resveratrol (i.e., tetramethoxystilbene (see Park et al. Cancer Res 67:5717-5726, 2007)) or naturally occurring resveratrol-like compound (i.e., a resveratrol analog (see Balestrazzi et al. Can J Microbiol 55:829-840, 2009)).

Exemplary polymers that the polyphenols and/or thyroid hormone analogs can be conjugated to include, but are not limited to polyvinyl alcohol, acrylic acid ethylene co-polymer, polyethyleneglycol (PEG), polyacrylic acid, polylactic acid, agarose, polyglycolide, polylactide, PEO, m-PEG, PVA, PLLA, PGA, poly-L-lysise, Human Serum Albumin, cellulose derivatives, carbomethoxy/ethyl/hydroxypropyl, hyaluronic acid, folate linked cyclodextrin/dextran, sarcosine/amino acid spaced polymer, alginate, carrageenan, pectin/chitosan, chitosan, dextran, collagen, polyamine, poly aniline, poly alanine, polytrytophan, poly tyrosine, or polylactide-co-glycolide (PLGA) having different molecular weights ranging from 2,000-20,000 Dalton. Other suitable polymers include, by way of non-limiting example, polyglycolide, polylactide, or copolymers thereof.

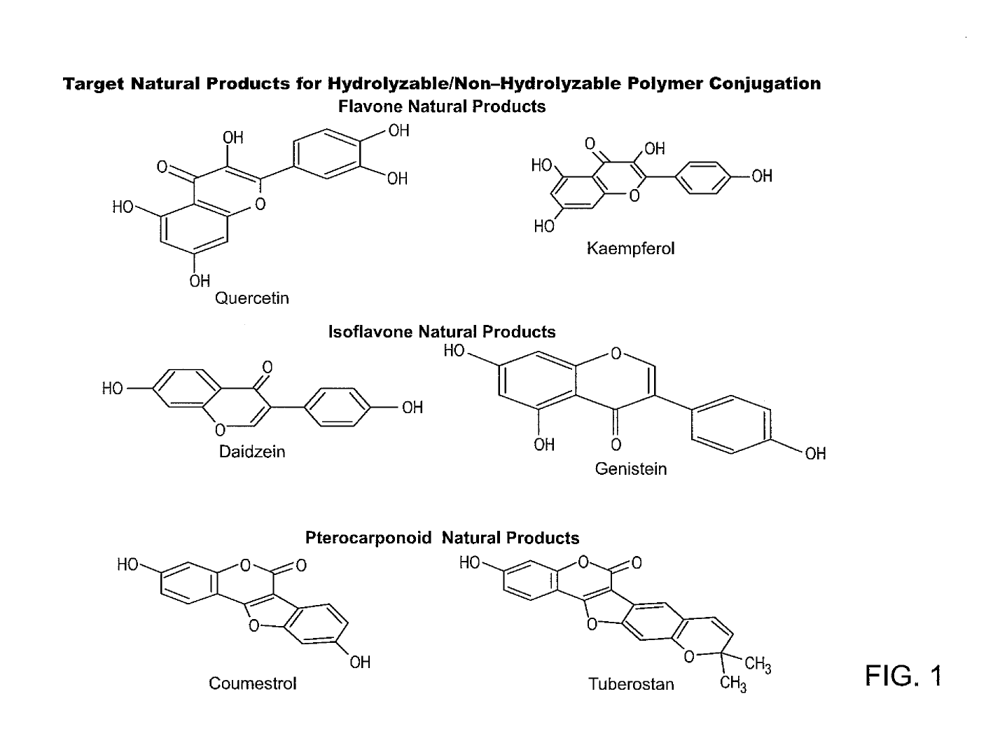

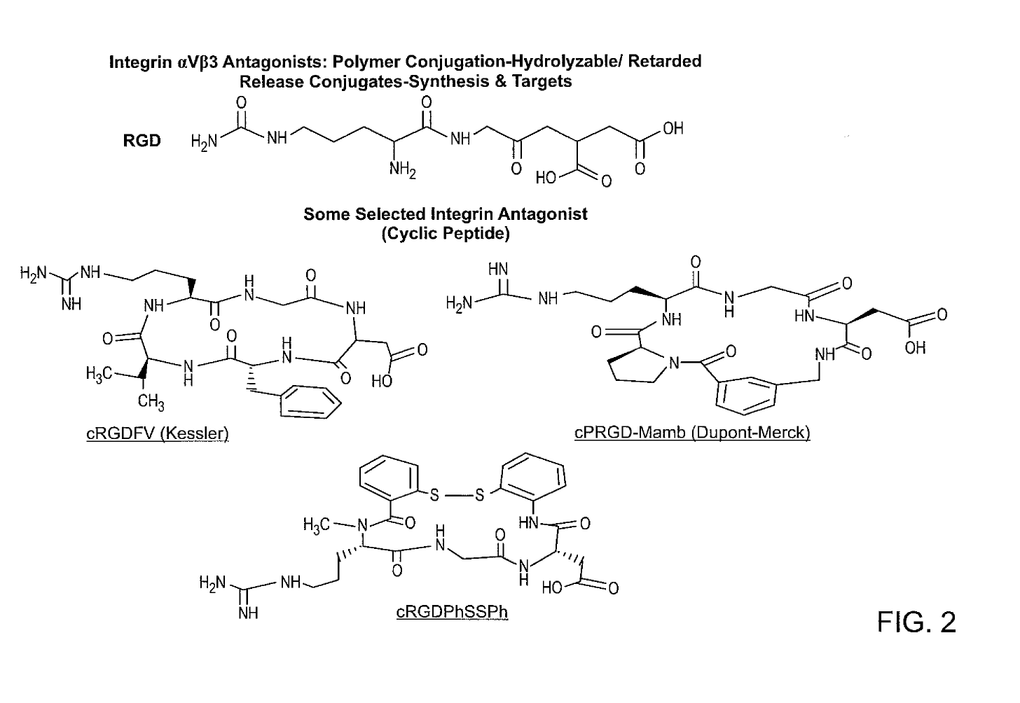

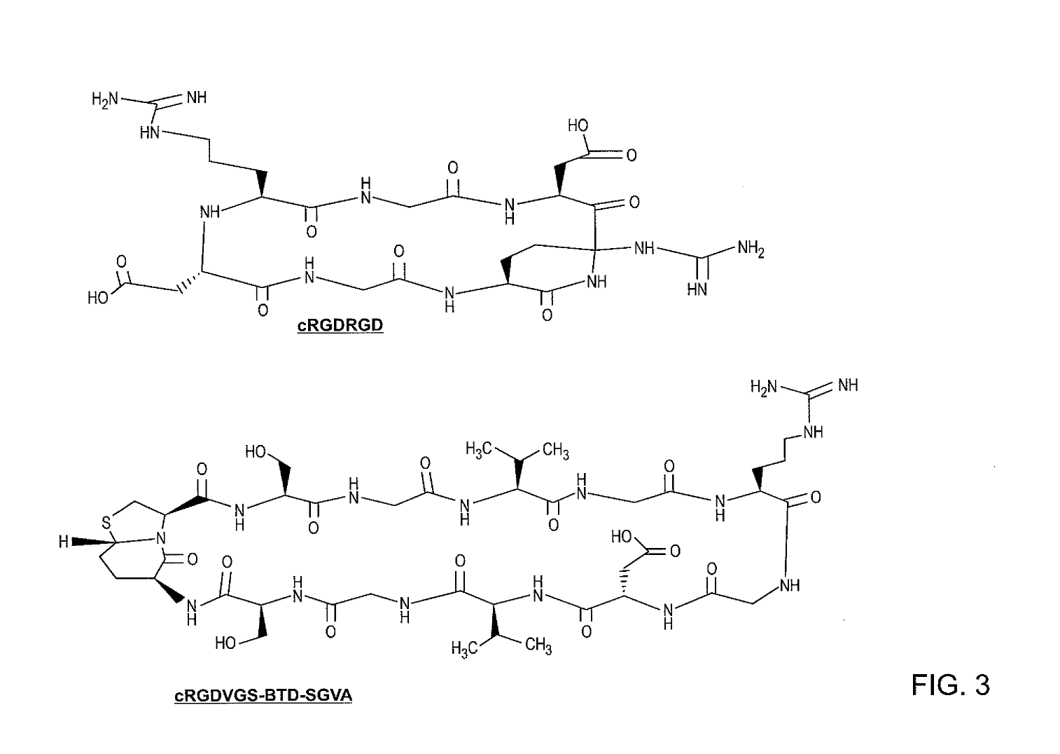

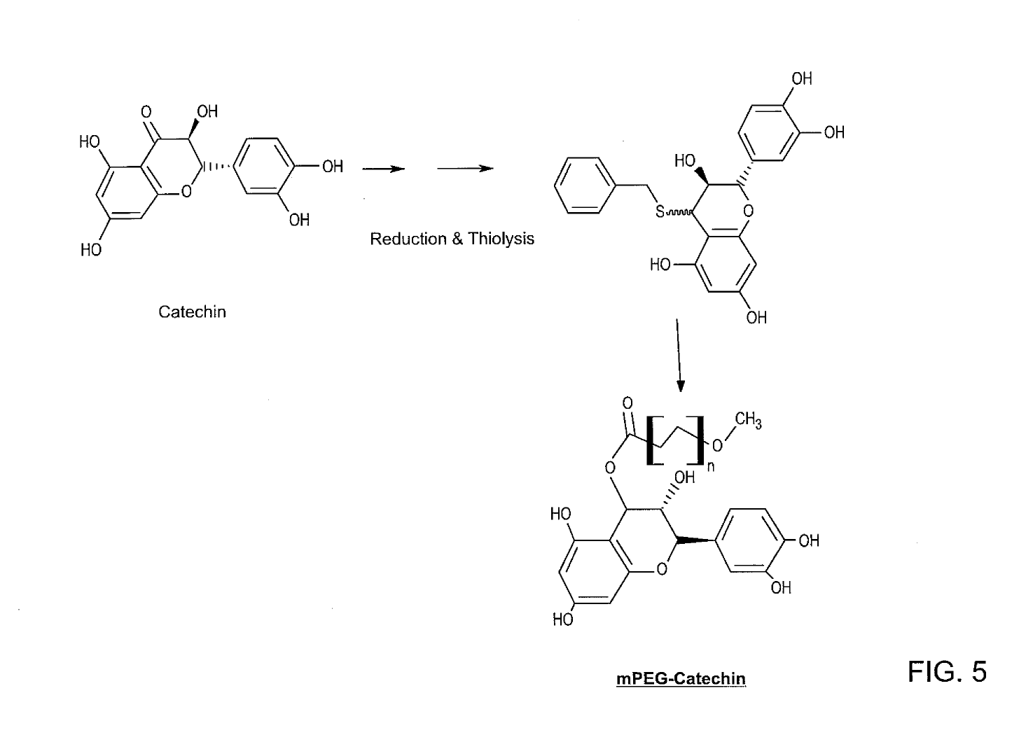

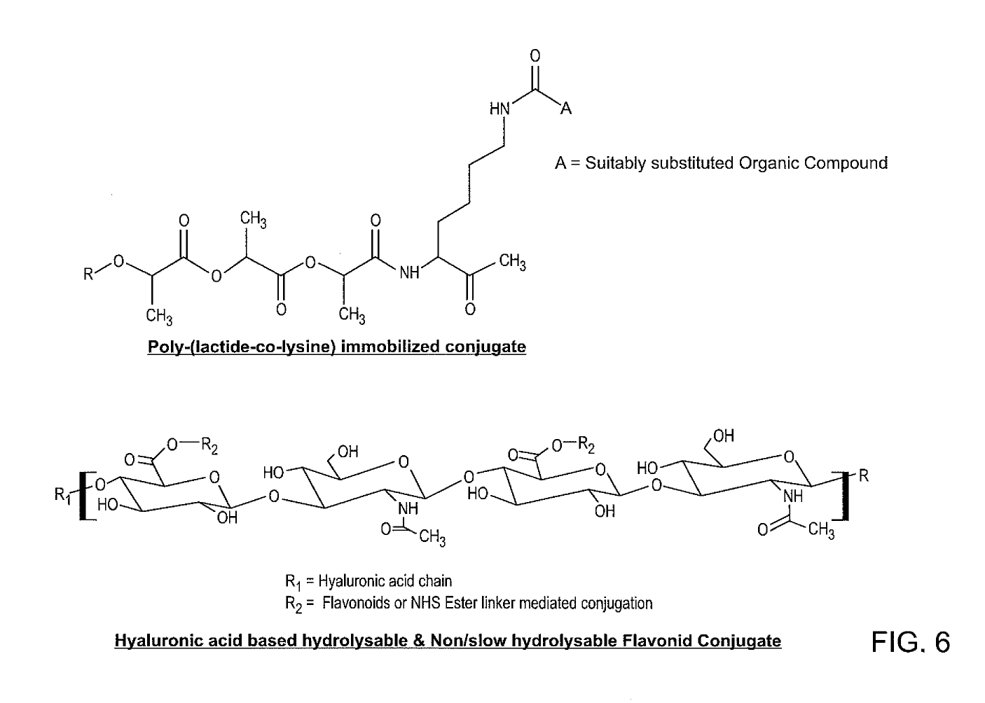

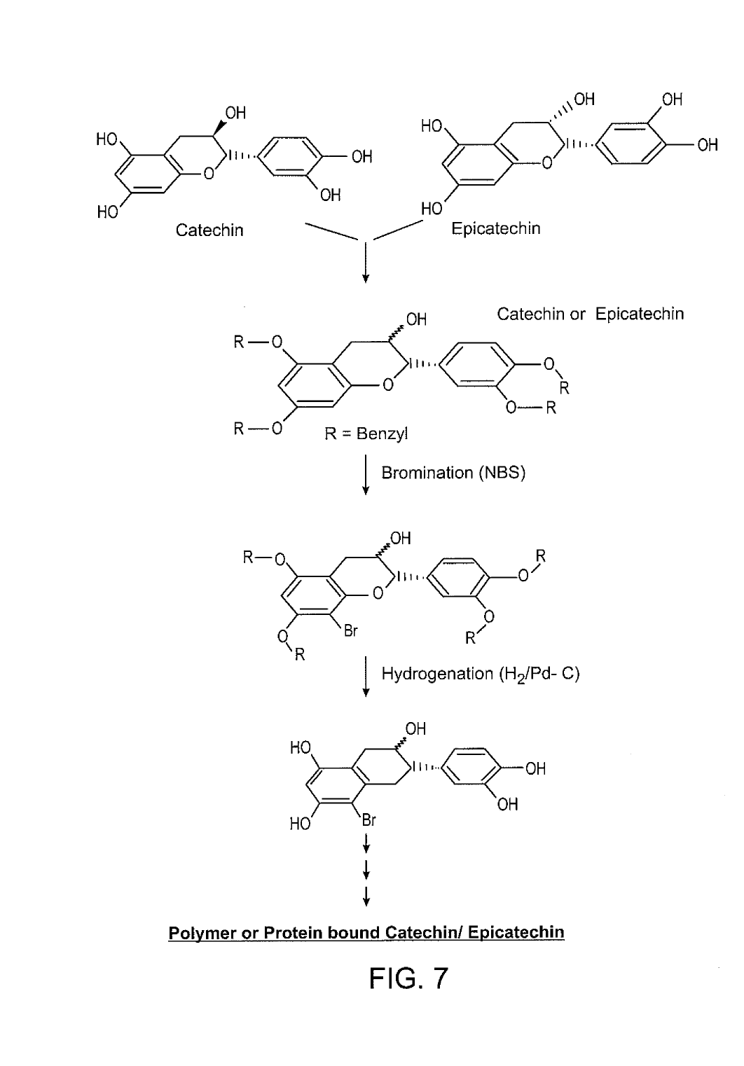

The RGD-binding compounds disclosed herein can also be used for preventing fat cell differentiation and/or fat accumulation (see Rayalam et al. Phyto Ther Res 22:1367-1371, 2008; Yang et al. Life Sci 82:1032-1039, 2008). In addition to polymer conjugated resveratrol, or analogs thereof, other suitable RGD-binding compounds can include, for example, RGD, and analogs or polymers thereof (See FIGS. 1-7). In one embodiment, a polymer-conjugated RGD analog can include another integrin antagonist that inhibits angiogenesis, such as, for example (but not limited to) EMD270179 (see Trieth et al. Int J Cancer 119:423-431, 2008) or trimestatin (see Fujii et al. J Mol Biol 332:1115-1122, 2003).

The structures of various RGD-binding compounds and formulations contemplated by the present invention are shown in FIGS. 1-7.

The pro-apoptotic action of resveratrol in cancer cells has been documented by a number of laboratories, including H. Y. Lin, P. J. Davis, F. B. Davis at Ordway (see Lin et al., J Urol 168:748-755, 2002; Lin et al. J Cell Biochem 104:2131-2142, 2008) and in collaboration with SA Mousa, Albany College of Pharmacy (see Lin et al. Cell Cycle 8:1877-1882, 2009). The critical contribution of the resveratrol receptor on integrin αvβ3 to induction of apoptosis was first shown in human breast cancer cells (see Lin et al. FASEB J 20:1742-1744, 2006). RGD peptide, but not control RGE peptide, and anti-integrin αvβ3 blocked activation of ERK1/2 by resveratrol and, downstream, resveratrol-induced, p-53-dependent apoptosis in human breast cancer (MCF-7 and MDA-MB-231) cells (see Lin et al. ibid.).

Resveratrol can be reformulated into a nanoparticle (e.g., by conjugation to a polymer). Conjugation of any of the compounds described herein can be accomplished via a covalent bond, e.g., an anhydride bond, an ester bond, an ether bond, or a sulfur linkage or another construct that limits action of the polyphenol to the cell surface receptor. Those skilled in the art will recognize that such reformulation prevents transport of the agent into the cell. The use of resveratrol nanoparticles is contemplated because their actions are limited to the integrin receptor for the polyphenol and they increase the half life of resveratrol. Thus, they represent novel structures of choice for induction of apoptosis in cancer cells. For example, non-GMP quality nanoparticulate resveratrol is synthesized. Those skilled in the art will also recognize that the fabrication and use of nanoparticulates of all polyphenol analogs of resveratrol can be used to induce programmed cell death in cancer cells via the integrin receptor for this polyphenol.

The steps by which resveratrol induces apoptosis in a variety of cancer cells, including glioma cells (see H Y Lin et al. Carcinogenesis 29:62-69, 2008) and head-and-neck cancer cells (see H Y Lin et al. J Cell Biochem, 104:62-69, 2009) are known. Moreover, it is now clear that a novel resveratrol-inducible pool of nuclear cyclooxygenase-2 (COX-2) is a transient, upstream participant in the p53-dependent apoptosis caused by the polyphenol (see H Y Lin et al. J Cell Biochem, ibid.; H Y Tang et al. Mol Cancer Ther 5:2034-2042, 2006). Likewise, COX-2 siRNA treatment of cancer cells inhibits induction of apoptosis by resveratrol. This inducible form of COX-2 contrasts with the constitutively expressed pool of COX-2 in many cancer cells, which is a marker of tumor cell aggressiveness. Without entering the cell, nanoparticulate resveratrol will induce the pool of nuclear COX-2 that fosters p53-dependent apoptosis in cancer cells. (See Tang et al. ibid.).

Resveratrol, via induction of ERK1/2-dependent Ser-15 phosphorylation of mutated p53, salvages the pro-apoptotic function of the latter (see HY Lin et al. J Urol 168:748-755, 2002). Although p53-independent apoptosis pathways are described, programmed cell death (apoptosis) in cancer cells is largely a p53-requiring process. Fifty percent of cancer cells have mutations in the p53 gene that prevent apoptosis. Thus, the novel observation that resveratrol rescues the pro-apoptotic function of mutated p53 (provided that Ser-15 is not a mutation site) is an important therapeutic asset of the polyphenol that renders treatable those cancers bearing mutations in p53.

The exposure of human breast cancer (MDA MB 231) cells in vitro to resveratrol for 6 days (with daily refreshment) of the polyphenol results in an anti-proliferative (pro-apoptotic) IC₅₀ of the polyphenol of 0.1 μM. This is 10-100 times lower than the anti-proliferative concentrations of resveratrol reported by other laboratories. (See Rayalam et al. Phytother Res 22:1367-1371, 2008 and Yang et al. Life Sci 82:1032-1039, 2008).

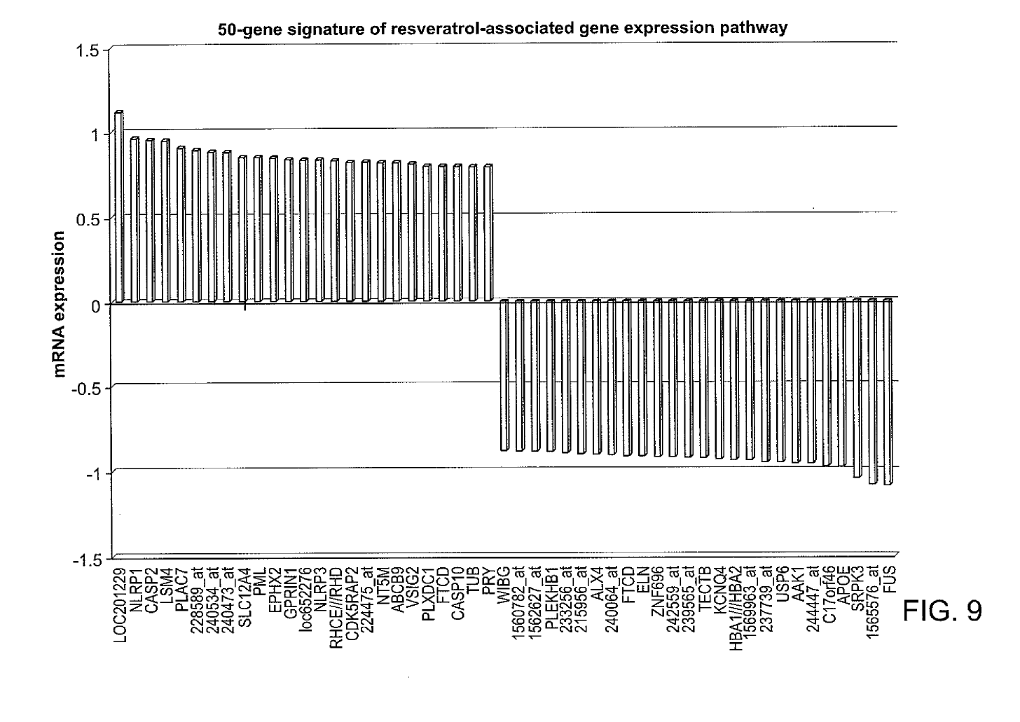

The gene targets for the pro-apoptotic action of resveratrol have also been identified as CASP2 and NALPJ (NLRPJ). (See FIG. 9). These specific gene targets for the anti-apoptotic action of resveratrol are downstream of the integrin receptor for the polyphenol. The interactions of the gene products with one another are complex, but understandable in the context of (resveratrol-induced) apoptosis. Among the cancer-relevant genes whose expression is downregulated by resveratrol (FIG. 9) are leukemia-associated FUS, a survival factor gene for colorectal cancer cells (AAKJ), and the USP6 (Tre6) oncogene. Other non-cancer related genes shown in FIG. 9 to be targets of resveratrol are related to the proposed beneficial effects of the polyphenol on nerve cell function and neurodegeneration.

It has been established that certain endogenous factors, such as epidermal growth factor (EGF) (see A Shih et al. Mol Cancer Ther 3:1355-1363, 2004), thyroid hormone (see H Y Lin et al. Steroids 72:180-187, 2007) and estrogen (see S Zhang et al. Br J Cancer 91:178-185, 2004), can, in physiological concentrations, block the pro-apoptotic activity of resveratrol. Accordingly, clinical trials of polyphenols must also take into account anti-apoptotic roles played by such endogenous hormones and growth factors.

Tetraiodothyroacetic acid (tetrac) is a thyroid hormone derivative with anti-proliferative activity in cancer cells that are initiated at a cell surface receptor for thyroid hormone on integrin αvβ3. (See P J Davis et al. Am J Physiol 297:E1238-E1246, 2009; H Y Lin et al. Am J Physiol 296:C980-C991, 2009; M Yalcin et al. Anticancer Res 10:3825-3831, 2009; A B Glinskii et al. Cell Cycle 8:3554-3562, 2009). Like resveratrol, tetrac is pro-apoptotic (see A B Glinskii et al., ibid.). Tetrac is also a polyfunctional anti-angiogenic agent, blocking the pro-angiogenic actions of T4 and T3 (see F B Davis et al., Circ Res 94:1500-1506, 2004), now known to be initiated at the integrin receptor, but also inhibiting the angiogenic effects of vascular endothelial growth factor (VEGF), basic fibroblast growth factor (bFGF) (see S A Mousa et al., Angiogenesis 11:183-190, 2008) and epidermal growth factor (EGF). Tetrac is a cancer chemosensitizing agent (see S A Mousa et al., Angiogenesis 11:269-276, 2008) and has been shown to radiosensitize glioma cells (see A Hercbergs et al., Cell Cycle 8:2586-2591, 2009).

Because the receptor site for thyroid hormone on this integrin is wholly independent of the resveratrol binding site (receptor) on the same protein, the combination of tetrac and resveratrol is more effective than either agent, alone, as anti-proliferative, pro-apoptotic agents. As such, the combination of resveratrol and tetrac (or any other anti-angiogenic thyroid hormone analog) exhibits a synergistic effect. Therefore, the combination of unmodified (i.e., non-polymer conjugated) resveratrol and tetrac, of nanoparticulate resveratrol and nanoparticulate tetrac, or of some combination thereof, represents a novel family of therapeutic modalities, which will eliminate the possibility that the anti-cancer action of resveratrol will be antagonized by endogenous (host) thyroid hormone. This action of endogenous thyroid hormone is a deficiency of the current clinical applications of resveratrol, alone, as a cancer chemotherapeutic agent. According to the methods and compositions of the invention, tetrac can also be used in conjunction with various polyphenol analogs of resveratrol as a chemotherapeutic agent. By way of non-limiting example, suitable polyphenols include flavonoids (e.g., flavones, catechins, flavanones and anthacyanidins) and isoflavonoids.

Additionally, it will be readily apparent to those skilled in the art that resveratrol and resveratrol analogs, whether unmodified or in nanoparticle form , can be used in combination with other anti-angiogenic thyroid hormone analogs, such as, for example, triiodothyroacetic acid (triac), the deaminated analog of 3,5,3′-triiodothyronine (T₃). Again, triac can be used either unmodified or as a nanoparticulate.

The combination of modified or unmodified resveratrol and modified or unmodified tetrac in a vehicle, for example, a single nanoparticle, can also be used together with one or more additional conventional cancer chemotherapeutic agents as a delivery system to target cancer cells for the additional chemotherapeutic agents. That is, the tetrac and the resveratrol are recognized and liganded by integrin αvβ3-bearing tumor cells, and this fact can be used to bring the tumor cells into contact with the additional chemotherapeutic agents. Thus, in this model, three (or more) anti-cancer agents are transported to the cancer cell.

It should be noted that the expression of integrin αvβ3 is principally observed on all tumor cells studied so far, endothelial cells, vascular smooth muscle cells and osteoclasts (see Davis et al. Am J Physiol 297:E1238-E1246, 2009). Hence, this proposed delivery system is relatively specific for cancer cells as well as the rapidly growing blood vessels that support tumors.

Those skilled in the art will recognize that all polyphenol analogs of resveratrol, whether unmodified or as nanoparticulates, in conjunction with tetrac or triac, whether unmodified or as nanoparticulates, in combination with one or more conventional chemotherapeutic agents can be fabricated and used.

Thus, combination of tetrac and resveratrol, whether unmodified or modified as a nanoparticulate, represents a novel treatment for the induction of apoptosis in cancer cells. Moreover, the combination excludes the antagonistic action of host endogenous thyroid hormone on the pro-apoptotic action of resveratrol. (See Lin et al. Carcinogenesis 29:62-69, 2008). Likewise, an anti-estrogen compound can be used in conjunction with the combination of tetrac and resveratrol, e.g., in estrogen-bearing breast cancers or lung cancers that are ER-positive. (See Koutras et al. Mol Cancer 8(1):109, 2009). That is, the combination of agents is directed at multiple sites of vulnerability in such cancer cells.

Additionally, the combination of resveratrol and tetrac in a vehicle, such as a nanoparticle, in further combination with one or more conventional cancer chemotherapeutic agents, permits delivery of such chemotherapeutic agents directly to integrin αvβ3-bearing tumor cells. Suitable chemotherapeutic agents include, but are not limited to, doxorubicin, etoposide, cyclophophamide, 5-fluoracil, cisplatin, trichostatin A, paclitaxel, gemcitabine, taxotere, cisplatinum, carboplatinum, irinotecan, topotecan, adrimycin, bortezomib, and atoposide or any combinations or derivatives thereof.

The RGD-binding compounds described herein are preferably administered in a formulation (including the analogs, polymeric forms, and/or any derivatives thereof) together with a pharmaceutically acceptable carrier. Any formulation or drug delivery system containing the active ingredients, which is suitable for the intended use, that are generally known to those of skill in the art, can be used. Suitable pharmaceutically acceptable carriers for oral, rectal, topical, or parenteral (including subcutaneous, intraperitoneal, intramuscular and intravenous) administration are known to those of skill in the art. Those skilled in the art will recognize that the carrier must be pharmaceutically acceptable in the sense of being compatible with the other ingredients of the formulation and not being deleterious to the recipient thereof.

Formulations suitable for parenteral administration may include sterile aqueous preparations of the active compound, which are preferably isotonic with the blood of the recipient. Thus, such formulations may contain distilled water, 5% dextrose in distilled water or saline. Useful formulations may also include concentrated solutions or solids containing any of the compositions or compounds described herein, which upon dilution with an appropriate solvent, give a solution suitable for parental administration.

For enteral administration, a compound can be incorporated into an inert carrier in discrete units such as capsules, cachets, tablets, or lozenges, each containing a predetermined amount of the active compound(s); as a powder or granules; or a suspension or solution in an aqueous liquid or non-aqueous liquid, e.g., a syrup, an elixir, an emulsion or a draught. Suitable carriers may be starches or sugars and may include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base. Alternatively, the compound may be administered in liposomes, microspheres (or microparticles), or attached to nanoparticles. Methods for preparing liposomes and microspheres for administration to a patient are well known to those of skill in the art. For example, U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 287-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

Any of the RGD-binding compounds, tetrac or triac, and/or the polymeric forms thereof can be formulated into nanoparticles. Preferred nanoparticles are those prepared from biodegradable polymers, such as, for example, polyethylene glycols, polyglycolide, polylactide and copolymers thereof. Those of skill in the art can readily determine an appropriate carrier system (i.e., the polymer) used for preparation of nanoparticulate forms of resveratrol and/or tetrac (or triac) depending on various factors, including, for example, the desired rate of drug release and the desired dosage.

In some embodiments, the formulations are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

The formulations may also be presented in unit dosage form and may be prepared by any of the methods well known in the art of pharmacy. For example, such methods include the step of bringing the active compound(s) into association with a carrier, which constitutes one or more accessory ingredients. In general, the formulations are prepared by uniformly and intimately bringing the active compound(s) into association with a liquid carrier or a finely divided solid carrier and then, if necessary, shaping the product into desired unit dosage form.

The formulations can optionally include one or more additional components, such as various biologically active substances including growth factors (e.g., including TGF-β, basic fibroblast growth factor (FGF2), epithelial growth factor (EGF), transforming growth factors α and β (TGF α and β), nerve growth factor (NGF), platelet-derived growth factor (PDGF), and vascular endothelial growth factor/vascular permeability factor (VEGF/VPF)), antivirals, antibacterials, anti-inflammatories, immuno-suppressants, analgesics, vascularizing agents, and/or cell adhesion molecules.

In addition, any of the formulations of the invention may further include one or more optional accessory ingredient(s) routinely utilized in the art of pharmaceutical formulations, e.g., diluents, buffers, flavoring agents, binders, surface active agents, thickeners, lubricants, suspending agents, and/or preservatives (including antioxidants) and the like.

The invention will be further illustrated in the following non-limiting examples.

### Examples

### Example 1

### Size Measurement of Resveratrol Doped poly(lactic-co-glycolic acid) (PLGA) or poly(lactic-co-glycolic acid)/polyvinyl alcohol (PLGA/PVA) Nanoparticles by Dynamic Light Scattering (DLS)

Resveratrol nanoparticles were prepared. The size of the nanoparticles was determined by DLS (single emulsion/solvent evaporation method), and ranged from 100-300 nm. In some embodiments, the surface of PLGA nanoparticles was coated with chitosan and/or PVA.

### Example 2

### Effect of Tetrac on Resveratrol-Induced Anti-Proliferation

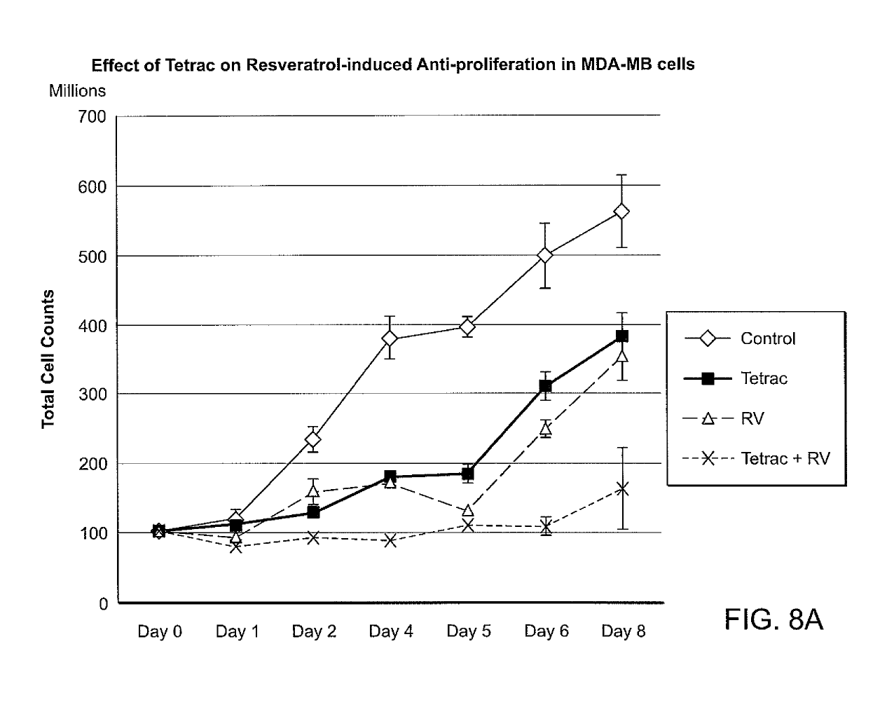

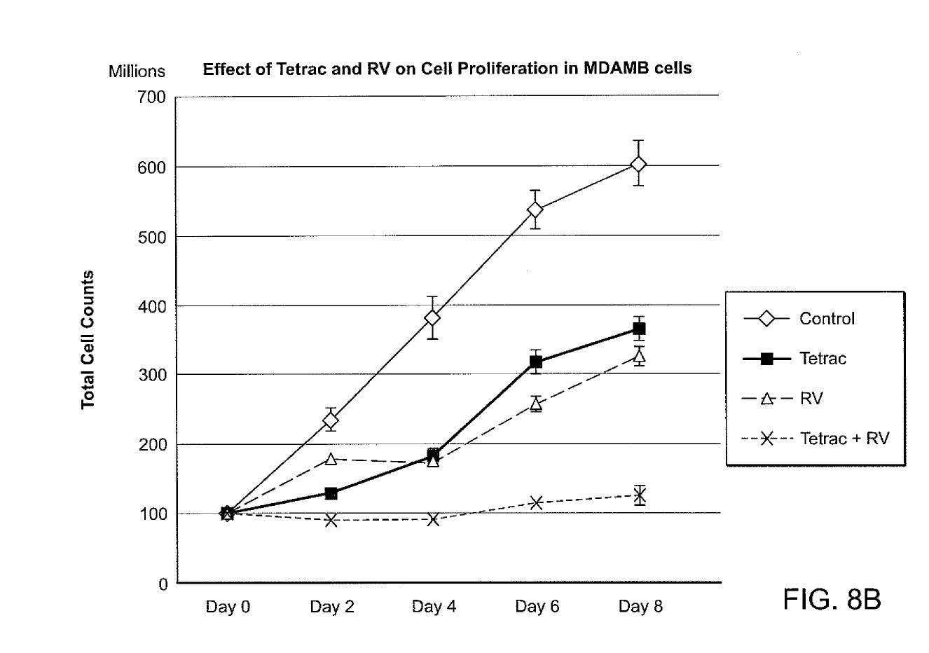

MDA-MB cells were seeded in perfusion CESCO bellow bottles. Cells were refreshed with 1% FBS containing DMEM after 24 h incubation. Cells were then treated with tetrac (10⁻⁷ M), or resveratrol (0.1 mM) or both. Resveratrol was added daily and tetrac was added every three days. Cells were harvested as indicated dates and counted. Results represented the average of 4 experiments are shown in FIG. 8. A similar experiment was run to determine the effect of tetrac and resveratrol on cell proliferation in MDA-MB cell, with similar results.

### Example 3

### Establishing Effectiveness of Resveratrol Nanoparticles in Inducing of Apoptosis in Cancer Cells in vitro

Comparison is made of unmodified resveratrol and resveratrol nanoparticles (10⁻⁷ to 10⁻⁵ M) on the induction of p53-dependent apoptosis in cancer cells with and without mutated p53. Cancer cell lines that are examined include human breast (MCF-7, MDA MB 231) cells, human prostate (LNCaP, DU-145) cells, glioblastoma (U87 MG) cells, small cell lung cancer (HTB 184) cells, follicular thyroid cancer (FTC 236) cells and adenoid cystic carcinoma salivary gland tumor (TGS112T) cells. The endpoints of the study may include PCNA production, radiolabeled thymidine incorporation, cell counts, cell sorting analysis for cell cycle arrest, and TUNEL assay for estimation of apoptosis.

### Example 4

### Comparison of the Results of Example 3 with a Combination of Resveratrol Nanoparticles or Unmodified Resveratrol with Tetrac Nanoparticles

Dosing for the polyphenol is as described in Example 3 with a combination of nanoparticulate tetrac dosing of 10⁻⁸ to 10⁻⁶M. Cells that are studied are MCF-7, LnCaP, U87 MG and HTB 184.

### Example 5

### Implicating the Integrin Receptor for Resveratrol in Cellular Actions of the Polyphenol on Sirtuins

Quantitation of sirtuin-mediated actions of resveratrol in mammalian cells (INK4 accumulation, acetylation of p53, etc.) in the absence and presence of RGD peptide, RGE, antibody to integrin αvβ3 is studied and carried out with DNA microarray comparisons in resveratrol-treated and untreated noncancer cells, e.g., human dermal fibroblasts, with microarrays from studies of resveratrol-treated cells published by others (see M Stefani et al. Ann NY Acad Sci 1114:407-418, 2007).

### Example 6

### Determination of Possible Contribution of Sirtuins on Cancer Cell Proliferation; Actions of Resveratrol Nanoparticles in the Sirtuin-Containing Cancer Cell and in Cancer Cells with Knockdown of SIRT1

siRNA for SIRT1 is used to reduce SIRT1 levels in MCF-7 and HTB 184 cells. Growth characteristics (doubling time) of untreated cells is then determined and cells are thereafter treated with resveratrol nanoparticles, tetrac nanoparticles or the combination. These studies define whether SIRT1 is a useful target of resveratrol in selected cancer cells.

### Example 7

### DNA Microarray Analysis of Cancer Cells Treated with Resveratrol Nanoparticles and a Combination of Nanoparticulate Tetrac and Resveratrol

DNA is harvested from control and treated MCF-7, HTB 184 and LNCaP cells for microarray (Functional Genomic Core Facility at Ordway Research Institute, Inc.) and interpreted. Microarray data has been generated for resveratrol alone (see FIG. 9), for tetrac alone, as well as for tetrac nano alone (see, e.g., U.S. Ser. No. 61/187,799, which is incorporated by reference in its entirety).

### Example 8

### Establishing the Effectiveness of Resveratrol Nanoparticles in Decreasing Cancer Cell Proliferation in Human Cancer Xenografts in Nude Mice

The effectiveness of unmodified resveratrol is compared with resveratrol nanoparticles in two tumor cell lines (human breast and prostate cancers) and results are obtained in castrated and intact female animals (to confront the issue of estrogen antagonism of resveratrol action) and in animals treated with antibody to the EGF receptor (to confront the issue of EGF antagonism of resveratrol action). A separate set of studies is considered in which thyroidectomy has been carried out to eliminate the possible anti-apoptotic effect of thyroid hormone.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pharmaceutical composition for treating cancer comprising a combination of small molecule ligand of the integrin RGD recognition site and an anti-angiogenic thyroid hormone analog, wherein the combination induces apoptosis in cancer cells.

2. The pharmaceutical composition of claim 1, wherein the cancer cells are selected from the group consisting of: breast cancer, lung cancer, kidney cancer, thyroid cancer, brain cancer (glioma), ovarian cancer, pancreatic cancer, prostate cancer, plasma cell cancer (myeloma), squamous cell head-and-neck cancer, liver cancer, muscle cancer (sarcoma), and colon cancer.

3. The pharmaceutical composition of claim 1, wherein the small molecule ligand of the integrin RGD recognition site is obtained or derived from a polyphenol compound and the thyroid hormone analog is tetrac or triac (triiodothyroacetic acid).

4. The pharmaceutical composition of claim 3, wherein the small molecule ligand of the integrin RGD recognition site is resveratrol.

5. The pharmaceutical composition of claim 4, wherein the thyroid hormone analog is tetrac.

6. The pharmaceutical composition of claim 5, wherein resveratrol, or tetrac or both are conjugated via a covalent bond to a polymer selected from polyvinyl alcohol, acrylic acid ethylene co-polymer, polyethyleneglycol (PEG), polyacrylic acid, polylactic acid, agarose, polyglycolide, polylactide, PEO, m-PEG, PVA, PLLA, PGA, poly-L-lysine, Human Serum Albumin, cellulose derivatives, carbomethoxy/ethyl/hydroxypropyl, hyaluronic acid, folate linked cyclodextrin/dextran, sarcosine/amino acid spaced polymer, alginate, carrageenan, pectin/chitosan, chitosan, dextran, collagen, polyamine, poly aniline, poly alanine, polytrytophan, poly tyrosine, polylactide-co-glycolide (PLGA) polyglycolide, polylactic acid, or co-polymers thereof, wherein said polymer is formulated into a nanoparticle, wherein said nanoparticle is between 150 and 250 nanometers in size, and wherein said tetrac binds to the cell surface receptor for thyroid hormone on integrin αvβ3.

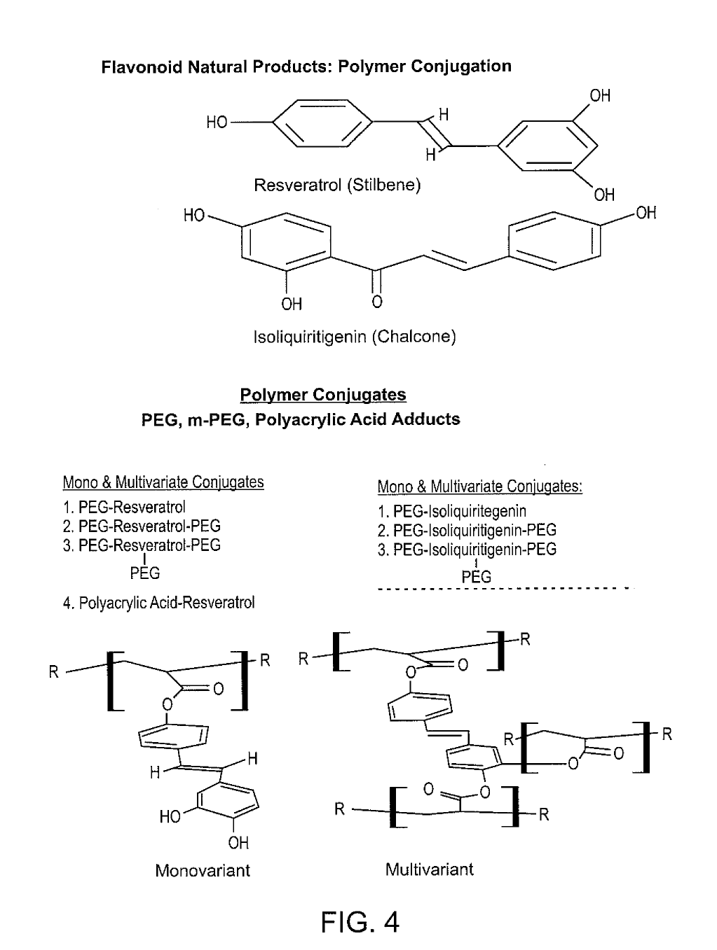

7. The pharmaceutical composition of claim 5, wherein resveratrol is formulated as any of the conjugates shown in FIGS. 4-7.

8. The pharmaceutical composition of claim 1, further comprising an anti-estrogen compound.

9. The pharmaceutical composition of claim 6, wherein the anti-estrogen compound is selected from the group consisting of tamoxifen and aromatase inhibitors.

10. The pharmaceutical composition of claim 6, wherein the nanoparticles further comprise one or more additional chemotherapeutic agents.

11. The pharmaceutical composition of claim 10, wherein the one or more additional chemotherapeutic agents are targeted to the cancer cells.

12. The pharmaceutical composition of claim 1, wherein the combination blocks anti-apoptotic action of endogenous thyroid hormone.

13. A method of treating cancer comprising administering a therapeutically effective amount of a combination of a small molecule ligand of the integrin RGD recognition site and an anti-angiogenic thyroid hormone analog to a patient suffering therefrom.

14. The method of claim 13, wherein the small molecule ligand of the integrin RGD recognition site is obtained or derived from a polyphenol compound and the thyroid hormone analog is tetrac or triac.

15. The method of claim 14, wherein the small molecule ligand of the integrin RGD recognition site is resveratrol.

16. The method of claim 15, wherein the thyroid hormone analog is tetrac.

17. The method of claim 16, wherein resveratrol or tetrac or both are conjugated via a covalent bond to a polymer selected from polyvinyl alcohol, acrylic acid ethylene co-polymer, polyethyleneglycol (PEG), polyacrylic acid, polylactic acid, agarose, polyglycolide, polylactide, PEO, m-PEG, PVA, PLLA, PGA, poly-L-lysine, Human Serum Albumin, cellulose derivatives, carbomethoxy/ethyl/hydroxypropyl, hyaluronic acid, folate linked cyclodextrin/dextran, sarcosine/amino acid spaced polymer, alginate, carrageenan, pectin/chitosan, chitosan, dextran, collagen, polyamine, poly aniline, poly alanine, polytrytophan, poly tyrosine, polylactide-co-glycolide (PLGA) polyglycolide, polylactic acid, or co-polymers thereof, wherein said polymer is formulated into a nanoparticle, wherein said nanoparticle is between 150 and 250 nanometers in size, and wherein said tetrac binds to the cell surface receptor for thyroid hormone on integrin αvβ3.

18. The method of claim 16, wherein resveratrol is formulated as any of the conjugates shown in FIGS. 4-7.

19. The method of claim 13, further comprising administering an anti-estrogen compound to the subject.

20. The method of claim 19, wherein the anti-estrogen compound is selected from the group consisting of tamoxifen and aromatase inhibitors.

21. The method of claim 17, wherein the nanoparticles further comprise one or more additional chemotherapeutic agents.

22. The method of claim 17, wherein the nanoparticles induce chemosensitization of chemoresistant cancer cells.

23. The method of claim 17, wherein the nanoparticles induce radiosensitivity or oppose radioresistence of cancer cells.
